(19) United States
(12) Patent Application Publication
(10) Pub. No.: US 2025/0078961 A1
(43) Pub. Date: Mar. 6, 2025

(54) METHOD OF HIGH THROUGHPUT SCREENING (HTS) FOR BIOACTIVES

(71) Applicant: ACCESS BUSINESS GROUP INTERNATIONAL LLC, Ada, MI (US)

(72) Inventors: Dmitri SITNIKOV, Greenville, MI (US); Sunghan YIM, Ada, MI (US); Joshua K. SCHWANNECKE, Grand Rapids, MI (US)

(21) Appl. No.: 18/823,328

(22) Filed: Sep. 3, 2024

Related U.S. Application Data
(60) Provisional application No. 63/536,476, filed on Sep. 4, 2023.

(51) Int. Cl. G16C 20/40; C12N 5/04; G01N 30/72; G01N 33/00; G16B 20/00

(52) U.S. Cl. CPC G16C 20/40; C12N 5/04; G01N 30/72; G01N 33/0098; G16B 20/00

(57) ABSTRACT

A screening method for the high-throughput detection of phytochemicals is provided. The method includes exposing an extract cell group to a plant extract to form an exposed cell group. Exposed signals generated by the extract cell group and control signals generated by the control group are measured and compared to generate a signal list consisting of exposed signals that satisfy an inclusion condition. The exposed signals of the signal list are compared to a list of pre-determined phytochemical signatures to generate a known phytochemical list and/or an unknown phytochemical list. The known phytochemical list includes phytochemicals having phytochemical signatures substantially matching one of the exposed signals of the signal list, and the unknown phytochemical list consists of all other phytochemicals. A chemical database comprising phytochemical associations with chemicals to generate a reactive chemical list for each phytochemical of the known phytochemical list.

### CROSS-REFERENCE TO RELATED APPLICATIONS

This application claims the benefit of U.S. Provisional Application 63/536,476, filed on 4 Sep. 2023, the disclosure of which is incorporated herein by reference in its entirety.

### FIELD OF THE DISCLOSURE

The subject disclosure generally relates to methods for screening compounds, and more specifically, to high throughput screening (HTS) methods for the detection of bioactive compounds (or molecules) in complex chemical mixtures, such as in plants, in plant extracts, or other complex samples such as biological fluids, etc.

### BACKGROUND OF THE DISCLOSURE

High Throughput Screening (HTS) of plants for bioactive compounds is a systematic and efficient approach used in drug discovery, agricultural research, and biotechnology to identify and isolate potentially valuable compounds from plant sources. This process involves the rapid testing of numerous plant extracts or compounds in a relatively short period of time to identify those with desirable biological activities, such as pharmaceutical, agricultural, or industrial applications.

The background of HTS for bioactive compounds from plants involves several key elements:
1) Natural Product Diversity: Plants are a rich source of diverse and complex chemical compounds that have evolved for various purposes, including defense against pests, pathogens, and environmental stressors. Many of these compounds possess bioactive properties that can be harnessed for various applications.
2) Traditional Medicine and Ethnobotany: Throughout history, indigenous cultures have utilized plant-derived compounds for medicinal purposes. Traditional medicine practices and ethnobotanical knowledge have provided a foundation for the discovery of bioactive compounds that can be validated through modern scientific methods.
3) Advancements in Technology: The development of high-throughput technologies, such as robotic systems, automated liquid handling, microplate readers, and sophisticated analytical techniques, has revolutionized the field of plant-based drug discovery. These technologies allow researchers to rapidly test thousands to millions of samples, making the screening process more efficient and cost-effective.
4) Assay Development: HTS requires the design and optimization of specific assays that can reliably measure the desired biological activity of interest. These assays may be based on biochemical, cell-based, or whole-organism systems, depending on the target and application.
5) Compound Libraries: HTS involves screening large collections of compounds, which can include natural extracts, purified compounds from plants, or synthetic chemical libraries. These libraries are carefully curated to encompass a wide range of chemical diversity and structural complexity.
6) Controlled chemical modification of compounds: HTS involves screening of a collection of compounds mentioned in the discussion of compound libraries above, that were modified by chemical, enzymatic or a combination of factors such as fermentation, biotransformation, etc.
7) Identification and Validation: During HTS, compounds showing promising bioactivity are referred to as “hits.” These hits undergo further testing to confirm and validate their activity. This often involves more detailed biological, chemical, and pharmacological analyses to ensure that the observed effects are reproducible and meaningful.
8) Chemical Characterization: Once validated, the active compounds need to be isolated and chemically characterized. Techniques such as chromatography (e.g., liquid chromatography (LC)), mass spectrometry (MS), and nuclear magnetic resonance (NMR) spectroscopy are used to identify the structure of the bioactive compounds.
9) Structure-Activity Relationship (SAR) Studies: Understanding the relationship between the chemical structure of a bioactive compound and its biological activity is crucial for optimization and further drug development. SAR studies involve making systematic changes to the compound's structure to assess the impact on its activity.
10) Application Areas: Bioactive compounds identified through HTS of plants have a wide range of potential applications, including drug discovery (for example, as lead compounds for pharmaceutical development), agrochemicals (pesticides and herbicides), nutraceuticals (functional foods and dietary supplements), and cosmetics.

Despite its potential, HTS of plants for bioactive compounds also faces challenges such as the complex mixture of compounds in plant extracts, the need for reliable assays that mimic biological systems, ethical considerations related to biodiversity conservation and traditional knowledge, and the time-consuming process of characterizing and developing leads into marketable products.

In summary, HTS of plants for bioactive compounds represents a powerful strategy that leverages the natural chemical diversity of plants to discover novel compounds with valuable applications across various industries. It combines traditional knowledge, modern technology, and interdisciplinary approaches to accelerate the discovery and development of bioactive molecules.

Another screening method is BDF (bioassay-directed fractionation), which starts with chromatography separation (or fractionation) of a mixture (e.g., a biological or plant preparation) and exposure of test material (e.g., cells or tissues) to such fractions to detect biological effect(s) of fraction material on cells or tissues. If a biological activity is detected, the molecular composition of the fraction is reviewed using mass spectrometry (MS) or other analytical methods. Candidate compounds which match physical, chemical, and biological properties of previously known ones and published elsewhere are selected for further investigations. Sec, e.g., U.S. Pat. No. 7,132,117 that describes BDF of Eurycoma longifolia.

As with conventional HTS methods, conventional BDF methods can also have one or more issues. For example, assignment of bioactivity to a particular compound is based on assumptions driven by the result of similarities in physical properties (molecular or monoisotopic mass, primarily) detected in the bioactive fraction and chemical properties of candidate compounds which properties published previously. In addition, the confirmation of bioactivity requires a validation of putative compounds via isolation and individual tests with a low success rate. This is generally due to a large false detection rate based on the presence of an enormous number of isobaric (the same monoisotopic mass) compounds with controversial biological activities in publications and databases. Therefore, the process of assigning confident compounds requires human intervention, which is lengthy and labor intensive.

Yet another screening method is a hollow fiber cell “fishing” method which uses cells attached to an assembly of hollow fibers. The assembly is exposed to a biological substrate such as plant extract. Then, the compounds are washed away from cells still being attached to fibers and analyzed using HPLC and a UV detector. The compounds that bind to the cells are detected by the difference in UV profile between cells exposed, i.e., are fished out.

That said, the hollow fiber method has several disadvantages. First, it is assessed using an analytical method with insufficient selectivity. Therefore, the identity of fished out substances cannot be validated against those in the plant extract. Second, the method detects only surface binding metabolites, because the extraction of cells from hollow fibers for the assessment of intracellular content is not possible. Finally, seeding and control of cell status in hollow fibers is very laborious.

Further, the high false assignment rate, on one hand and an intrinsic restriction to a possible number of detected bioactive molecules implied by a nature of a bioassay on another, also reduces the possible number of bioactive compounds detected in a single experiment. Moreover, the selection of candidate compounds is limited to particular bioassay(s) and is relevant to already existing (i.e., published) data. Therefore, the discovery of unknown phytochemicals is impossible.

In view of the foregoing, there remains an opportunity to provide improved screening methods that are less tedious, less time-consuming, more accurate, and more cost-effective. There also remains an opportunity to provide new phytochemicals and/or new uses thereof.

### BRIEF SUMMARY OF THE DISCLOSURE

A screening method for the high-throughput detection of phytochemicals is provided. The method comprises exposing an extract cell group to a plant extract comprising one or more phytochemicals to form an exposed cell group. Exposed signals associated with one or more phytochemicals and control signals generated by a control group are measured. The exposed signals and the control signals are compared to generate a signal list consisting of exposed signals that satisfy an inclusion condition. The exposed signals of the signal list are compared to a list of pre-determined phytochemical signatures to generate a known phytochemical list and/or an unknow phytochemical list. Each phytochemical signature is associated with an individual phytochemical. The known phytochemical list consists of phytochemicals having phytochemical signatures substantially matching one of the exposed signals of the signature list. The unknown phytochemical list consists of any of the one or more phytochemicals that correspond with the exposed signals that do not substantially match one of the exposed signals of the signature list. A chemical database comprising phytochemical associations with chemicals is searched to generate a reactive chemical list for each phytochemical of the known phytochemical list.

The subject disclosure generally relates to methods for screening compounds, and more specifically, to high throughput screening (HTS) methods for the detection of bioactive compounds (or molecules) in complex chemical mixtures, such as in plants, in plant extracts, etc.

The subject disclosure also relates to bioactive compounds, molecules, or phytochemicals screened via the methods of this disclosure. In addition, the subject disclosure also relates to compositions and uses of such bioactive compounds, molecules, or phytochemicals.

In various embodiments, the subject disclosure relates to experimental metabolomics methodology which discovers simultaneously multiple bioactive phytochemicals in complex mixtures such as plants.

In various embodiments, a method of the subject disclosure utilizes LC-MS (liquid chromatography connected to mass spectrometer) based detection of compounds which bind to cells due to their exposure to a complex mixture of chemicals (such as plant extracts), followed by automated assignment of their physiological function. Other LC-MS systems, similar LC systems and/or MS systems, or equivalent systems may also be utilized.

In general, the methodology starts from the exposure of cells cultured in vitro (but the subject disclosure is not limited to cell culture) to an extract (e.g., metabolomic or proteomic) from plant tissues. It should be appreciated that the subject disclosure is not limited to plant tissues.

Then, a plethora of signals generated by compounds in samples prepared from cells exposed to the extract are compared to signals in identical cells, which were not exposed to the extract (i.e., a negative control). The comparison generates a list of signals higher in the exposed cells vs. in the not exposed cells and are also present in the extract.

In certain embodiments, the signals are then assigned to known structures via MS and NMR based experiments. Known physiological and biological functions can then be assigned to identified compounds using a computerized application (or applications) in an automated or near-automated fashion. The application(s) may use, e.g., the NCBI database PubChem and/or the David bioinformatic resources, for the identification of detected compounds. Additionally, or alternatively, other resources for identification may also be utilized.

In further embodiments, the detected compounds are then isolated (e.g., fractionated) using chromatography approaches for further analysis such as biological or genetic assays. Various chromatography approaches understood in the art can be utilized.

In general, the method of this disclosure does not use assumptions driven by the result of bioassay tests and physical and chemical similarities to already known bioactive compounds. This allows a direct and confident detection of putative biologically or chemically active compounds.

In addition, the method of this disclosure provides the opportunity of a high-throughput identification of putative multiple compounds using (but not limited to) mass spectrometry (MS) and an automated, bioinformatic meta-analysis for all these compounds which assign biological activities to them. The method also provides for identifying numerous compounds in a single experiment, which ensures a high-throughput discovery of unknown (i.e., new) phytochemicals.

The follow embodiments are also provided. A compound screened via the method (or methods) described herein, use of the compound (or compounds), a composition comprising the compound (or compounds), and use of the composition (or compositions). For example, the composition can be an oral composition or a topical composition for delivering and/or administering one or more bioactive compounds to a subject.

These and other objects, advantages, and features of the disclosure will be more fully understood and appreciated by reference to the description of the current embodiment and the drawings. Before the embodiments of the present disclosure are explained in detail, it is to be understood that the disclosure is not limited to the details of operation or to the details of construction and the arrangement of the steps or components set forth in the following description or illustrated in the drawings.

### BRIEF DESCRIPTION OF THE DRAWINGS

Other advantages of the disclosure will be readily appreciated, as the same becomes better understood by reference to the following detailed description when considered in connection with the accompanying drawings wherein:

FIG. 1 is a flow chart providing an overview of a method of HTS of plants for bioactive phytonutrients.

FIG. 2 is a flow chart providing the method of differential analysis used to determine and identify phytochemicals.

FIG. 3 is an infographic showing compounds detected in mass spectrometry (MS) analysis of loquat (Eriobotrya japonica) leaves.

### DETAILED DESCRIPTION OF THE CURRENT EMBODIMENTS

In the following description, numerous specific details are set forth to provide a thorough understanding of the present disclosure. It will be apparent, however, to one having ordinary skill in the art that the specific detail need not be employed to practice the present disclosure. In other instances, well-known materials or methods have not been described in detail to avoid obscuring the present disclosure.

Reference throughout this specification to “one embodiment”, “an embodiment”, “one example” or “an example” means that a particular feature, structure, or characteristic described in connection with the embodiment or example is included in at least one embodiment of the present disclosure. Thus, appearances of the phrases “in one embodiment”, “in an embodiment”, “one example” or “an example” in various places throughout this specification are not necessarily all referring to the same embodiment or example. Furthermore, the features, structures or characteristics may be combined in any suitable combinations and/or sub-combinations in one or more embodiments or examples.

Enumeration may be used in the description of various embodiments. Unless otherwise expressly stated, the use of enumeration should not be construed as limiting the disclosure to any specific order or number of steps or components. Nor should the use of enumeration be construed as excluding from the scope of the disclosure any additional steps or components that might be combined with or into the enumerated steps or components.

As discussed herein, the current embodiments relate to a screening method (“the method”) for the high-throughput detection of phytochemicals. The method is based on the phenomena of binding chemicals to target entities (cells and/or constituents therein) to exert their effect. The method comprises exposing cells (an extract cell group) to a plant extract to form an exposed cell group. The plant extract comprises one or more phytochemicals. Exposed signals generated by the exposed cell group and control signals generated by the control cell group (not exposed to an extract) and, in some embodiments, extract signals associated with the plant extract are measured. The exposed signals are associated with the one or more phytochemicals of the plant extract. In certain embodiments, the extract signals are compared with phytochemicals of the plant extract itself and to the control signals. The signals that are higher in the exposed cell group than in the control group and associated with signals of plant extract form a signal list consisting of exposed signals that satisfy an inclusion condition. The exposed signals of the signal list are compared to a list of pre-determined phytochemical signatures to generate a known phytochemical list. Each phytochemical signature is associated with an individual phytochemical, and if they substantially matched, generate a known phytochemical list. Other signals form an unknown phytochemical list. Therefore, the known phytochemical list consists of phytochemicals having phytochemical signatures substantially matching one of the exposed signals of the signature list. The unknown phytochemical list consists of any of the one or more phytochemicals that correspond with the exposed signals that do not substantially match one of the exposed signals of the signature list. A chemical database comprising phytochemical associations with chemicals to generate a reactive list for each phytochemical of the known phytochemical list. Further, in particular embodiments, a plethora of chemical databases are searched to generate an activity list for each phytochemical from the activity list. These chemical databases comprise phytochemicals and their associations with known biological, physiological or health effects.

The method may comprise culturing a plurality of cells and subdividing the plurality of cells into the extract cell group and the control cell group. Generally, the extract cell group and the control cell group are each disposed within a cell media. This cell media provides essential nutrients, hormones, and growth factors that cells need to propagate. Common media include DMEM (Dulbecco's Modified Eagle Medium), RPMI-1640, and MEM (Minimum Essential Medium), which may be supplemented with fetal bovine serum (FBS) or other growth factors. A cell suspension was formed by detaching cells from an original source, usually a cell culture flask or a tissue sample, using enzymes like trypsin or mechanical methods. The cells are usually counted using a hemocytometer or an automated cell counter. The appropriate number of cells are then seeded into a culture dish or flask containing the cell media. The culture dish or flask containing the plurality of cells is incubated in an incubator at an incubation temperature (generally 37° C.) and maintained in a controlled environment with appropriate humidity and CO₂ levels (e.g., 5% CO₂). The plurality of cells are monitored and the cell media may be replaced to replenish nutrients and/or remove waste products.

The plurality of cells are not particularly limited, and may be taken from any organism or any tissue. In some embodiments, the plurality of cells may be taken from a multitude of organisms or tissues. Non-limiting examples of cells include cocci, bacilli, spirilla, vibrios, mycoplasma, methanogens, halophiles, thermophiles, neurons, skeletal muscle cells, cardiac muscle cells, smooth muscle cells, squamous epithelial cells, cuboidal epithelial cells, columnar epithelial cells, fibroblasts, adipocytes, chondrocytes, osteocytes, erythrocytes, neutrophils, lymphocytes, monocytes, cosinophils, basophils, platelets, endothelial cells, exocrine gland cells, endocrine gland cells, parenchyma cells, collenchyma cells, sclerenchyma cells, fiber cells, sclerids, tracheids, vessel elements, sieve tube elements, companion cells, epidermal cells, guard cells, trichomes, mesophyll cells, hyphal cells, yeast cells, spore cells, embryonic stem cells, hematopoietic stem cells, mesenchymal stem cells, neural stem cells, sperm cells, oocytes, amoeba cells, paramecium cells, euglena cells, chlorella cells, spirogyra cells, carcinomas, sarcomas, leukemias, and lymphomas.

The cell media is not particularly limited, and may be any suitable media for culturing and containing cells. Non-limiting examples of suitable cell media include Eagle's Minimal Essential Medium (MEM), Dulbecco's Modified Eagle Medium (DMEM), RPMI 1640, Ham's F-12, L-15 Medium, Basal Medium Eagle (BME), Opti-MEM, MCDB Media (Medium-199, MCDB 131), HAMS F-10, Coon's Modified RPMI (CM-RPMI), SFM (Scrum-Frec Medium), Dulbecco's Modified Eagle Medium/Nutrient Mixture F-12 (DMEM/F-12), Endothelial Cell Medium (ECM), Neurobasal Medium, Keratinocyte-SFM, Mesenchymal Stem Cell Medium, Mouse Embryonic Fibroblast (MEF) Medium, Hematopoictic Stem Cell Medium, Dendritic Cell Medium, Primary Cell Culture Media, McCoy's 5A Medium, Williams' E Medium, Leibovitz's L-15 Medium, F-12K Medium, Clyde's Medium, Trypsin-EDTA Solution, Phosphate Buffered Saline (PBS), Hanks' Balanced Salt Solution (HBSS), Grace's Insect Medium, SF900 II SFM, Murashige and Skoog (MS) Medium, Gamborg's B5 Medium, Fetal Bovine Serum (FBS) supplemented media, Horse Serum supplemented media, KnockOut DMEM, StemPro™ Medium, and TeSR™ Medium.

In some embodiments, the method further includes preparing a cell-free control and a media-free control. The cell-free control comprises plant extract and cell media. The media-free control comprises the plant extract. Both the cell-free control and the media-free control are substantially free, or optionally entirely free, of the plurality of cells. The media-free control is substantially free, or optionally entirely free, of the cell media. The phrase “substantially free” in this context means that the relative amount of cells in the cell-free control/media-free control is less than 5%, optionally less than 3%, optionally less than 1%, or optionally less than 0.1%, of the amount of cells in the extract cell group and the control cell group. It will be appreciated that the exposed cell group is differentiated from the control cell group predominantly by the presence of the plant extract. The cell-free control is differentiated from the exposed cell group predominantly by the absence of the plurality of cells. The media-free control is differentiated from the exposed cell group predominantly by the absence of the plurality of cells and the absence of the cell media. A comparison between the exposed cell group, the control group, the cell-free control, and the media-free control can isolate the relative contribution of the plurality of cells, the cell media, and the plant extract. In some embodiments, the method further comprises incubating the extract cell group, the control cell group, and the cell-free control. The method may also comprise the step of separating the extract cell group and the control cell group from the respective cell media. Separation of the extract cell group and/or the control cell group may be performed using enzymatic detachment or mechanical detachment of the cell media. In some embodiments, the method further comprises comparing the exposed signals to extract signals of the media-free control to associate the exposed signals with the one or more phytochemicals.

The method comprises the step of exposing an extraction cell group to a sample, which can originate from plants or other natural sources (for example, mineral deposits, animal products, biological fluids, etc.). A plant extract comprising one or more phytochemicals is delivered to form an exposed cell group. Generally, the step involves disposing the plant extract in the cell media for the extract cell group in an extract concentration. In some embodiments, the method further comprises preparing the plant extract. Preparing the plant extract includes harvesting fresh or dried plant materials including leaves, roots, seeds, or stems. The plant materials are cleaned by washing the plant material to remove dirt pesticides, or other contaminants. The plant material is then dried to reduce the amount of moisture. The plant material may be dried via air-drying, using a dehydrator, or an oven set to a low temperature. In certain embodiments, the plant material is ground into a fine powder to increase surface area and improve extraction efficiency. The plant extract is extracted from the plant material. Any suitable extraction method may be used. Non-limiting examples of extraction methods include maceration, Soxhlet extraction, ultrasonic extraction, steam distillation, fermentation, pre-digestion, and press extraction. If the plant extract is extracted from the plant material using a solvent, the solvent is removed from the extract using a rotary evaporator or other evaporation method. The plant extract may be filtered to remove any residual particulates.

The plant extract is not particularly limited, and may be any processed or raw plant material. Non-limiting examples of plant extracts include essential oils (e.g., lavender extract, peppermint extract, tea tree extract, eucalyptus extract, rosemary extract, chamomile extract, lemon extract, geranium extract, cedarwood extract, ylang-ylang extract); herbal extract (e.g., ginseng extract, echinacea extract, ginger extract, turmeric extract, Ginkgo biloba extract, saw palmetto extract, St. John's Wort extract, valerian extract, milk thistle extract, hawthorn extract); fruit extracts (e.g., aloe vera extract, pomegranate extract, green tea extract, blueberry extract, acai berry extract, cranberry extract, cucumber extract, apple extract, grape seed extract, lemon balm extract); seed extracts (e.g., flaxseed extract, chia seed extract, pumpkin seed extract, sunflower seed extract, hemp seed extract); root extracts (e.g., licorice root extract, astragalus root extract, burdock root extract, dong quai extract, angelica root extract); leaf extracts (e.g., nettle extract, dandelion extract, thyme extract, mint extract, oregano extract); flower extract (e.g., rose extract, jasmine extract, hibiscus extract, calendula extract, elderflower extract); bark extract (e.g., cinnamon extract, cinchona extract, Pau d′Arco extract, willow bark extract, slippery elm extract); fungi extracts (e.g., reishi mushroom extract, shiitake mushroom extract, cordyceps extract, chaga mushroom extracts); and other extracts (e.g., sea buckthorn berry extract, grapefruit seed extract, agarwood extract, neem leaf or seed extract).

The plant extract comprises one or more phytochemicals. Phytochemicals are not especially limited, and may be any chemical compound contained in the plant extract. Non-limiting examples of phytochemicals include polyphenols, terpenoids, alkaloids, glycosides, glucosinolate, saponins, lignans, alkylphenols, carotenoids, organosulfur compounds, and phytosterols. Examples of polyphenols include flavonoids (e.g., anthocyanins, flavonols, flavones, isoflavones, catechins), phenolic acids (e.g., caffeic acid, ferulic acid, chlorogenic acid), tannins (e.g., ellagitannins, catechins). Examples of terpenoids include monoterpenes (e.g., limonene, pinene), sesquiterpenes (e.g., beta-caryophyllene, humulenc), diterpenes (e.g., retinoids), and sesterterpenes (e.g., farnesol). Examples of alkaloids include morphine, caffeine, nicotine, quinine, and capsaicin. Examples of glycosides include saponins, cardiac glycosides, and flavonoid glycosides. Examples of glucosinolates include sulforaphane and indole-3-carbinol. Examples of saponins include ginsenoside and soyasaponins. Examples of lignans include secoisolariciresinol and matairesinol. Examples of alkylphenols include resveratrol and ellagic acid. Examples of carotenoids include beta-carotene, lutein, zeaxanthin, and lycopene. Examples of organosulfur compounds include allicin, diallyl disulfide, and glucosinolates. Examples of phytosterols include beta-sitosterol and campesterol.

The method further comprises measuring exposed signals associated with the one or more phytochemicals and generated by the exposed cell group and control signals generated by a control cell group. Generally, the step of measuring the exposed signals and the control signals involves the use of mass spectroscopy and/or nuclear magnetic resonance techniques. Mass spectrometry (MS) is an analytical technique used to measure the mass-to-charge ratio of ions. MS provides detailed information about the molecular weight and structure of the one or more phytochemicals. During MS, the sample is introduced into a mass spectrometer, cither in a liquid or gas phase, depending on the ionization method used. The sample molecules are ionized to create charged particles. These ions are then directed into a mass analyzer. The mass analyzer separates the ions based on their mass-to-charge ratio. Different analyzers use various methods to achieve this separation. The separated ions are detected, and their abundance is recorded. The detector generates a mass spectrum, which is a plot of ion intensity versus mass charge (m/z). The mass spectrum is analyzed to identify the compounds present in the sample and to determine their concentrations. Identification may involve comparing the spectrum to known standards or databases.

Nuclear Magnetic Resonance (NMR) is an analytical technique used to determine the structure, dynamics, reaction state, and chemical environment of molecules. NMR exploits the magnetic properties of certain atomic nuclei to provide detailed information about the molecular structure and interactions. The sample is dissolved in a suitable solvent (often deuterated to avoid interference) and placed in a sample tube. The sample is exposed to a strong magnetic field, causing the nuclear spins to align with the field. Radio frequency (RF) pulses are applied to the sample, flipping the nuclear spins from their lower energy state to a higher energy state. The RF pulse is turned off, and the nuclei relax back to their original state, emitting RF signals in the process. These signals are detected and converted into a spectrum. The resulting spectrum is analyzed to determine the chemical environment of the nuclei. Peaks in the spectrum correspond to different chemical environments and interactions, providing information about the molecular structure.

In some embodiments, the exposed signals and the control signals are mass spectroscopy and/or nuclear magnetic resonance signals. Mass spectroscopy signals are electrical signals generated by ions in the relevant samples, which are measured alongside the mass and quantity of the ions. NMR signals are electromagnetic signals in the radio frequency.

The method comprises comparing exposed signals and control signals to generate a signal list consisting of exposed signals that satisfy an inclusion condition. Generally, this step involves comparing the height or area of specific peaks to determine if the amount of the phytochemical is significantly different in the experimental sample compared to the control. The clarity of signals above background noise is assessed to validate that the exposed signals for the phytochemical is discernible and different from potential noise in the control signals. MS and/or NMR peaks are matched with known standards to confirm that the detected peaks correspond to an expected phytochemical. The amounts of the expected phytochemical are then measured and compared. Multivariate statistical analysis is performed to analyze patterns in the spectral data to identify differences between experimental and control samples based on overall spectral profiles and determine the presence of specific phytochemicals. Generally, the inclusion condition is satisfied when an individual exposed signal is greater than a corresponding individual control signal.

The method further includes comparing the exposed signals of the signal list to a list of pre-determined phytochemical signatures to generate a known phytochemical list and/or an unknown phytochemical list. Each phytochemical signature is associated with at least one individual phytochemical, and generally exactly one individual phytochemical. The phytochemical signature is generally predetermined and may be well known and/or published in scientific literature. In some embodiments, the phytochemical signatures are stored in a phytochemical signature database. The known phytochemical list consists of phytochemicals having phytochemical signature substantially matching one of the exposed signals of the signal list. The known phytochemical list consists of any of the one or more phytochemicals that correspond with the exposed signals that do not substantially match one of the exposed signals of the signal list. It will be appreciated that every measured phytochemical will either be on the known or unknown phytochemical list. The term “substantially match” means that the phytochemical signature and the exposed signal are statistically significant within traditional statistical measures (e.g., p-value of less than 0.05).

In certain embodiments, the method includes generating the unknown phytochemical list and further comprises isolating any of the one or more phytochemicals of the unknown phytochemical list from the exposed cell group and/or plant extract. Generally, isolating any of the one or more phytochemicals of the unknown phytochemical list comprises using chromatography to separate any of the one or more phytochemicals of the unknown phytochemical list from the exposed cell group and/or plant extract. In particular embodiments, the method comprises generating the known phytochemical list and isolating any of the one or more phytochemicals of the known phytochemical list from the exposed cell group and/or plant extract. Generally, isolating any of the one or more phytochemicals of the known phytochemical list comprises using chromatography to separate any of the one or more phytochemicals of the known phytochemical list from the exposed cell group and/or plant extract. A variety of chromatography techniques may be used, including thin-layer chromatography, column chromatography, high-performance liquid chromatography, and gas chromatography.

In some embodiments, the method further comprises characterizing any of the one or more phytochemicals of the unknown phytochemical list. Characterizing phytochemicals involves determining their chemical structure, properties, and biological activities. Non-limiting examples of characterization techniques include X-ray crystallography, antioxidant activity testing, antimicrobial activity testing, cytotoxicity and cell viability testing, reactivity tests, and enzyme inhibition testing. In specific embodiments, the step of characterizing any of the one or more phytochemicals of the unknown phytochemical list may further involve identifying any of the one or more phytochemicals of the unknown phytochemical list.

The method comprises computerized or non-computerized searching a chemical database comprising phytochemical associations with chemicals to generate a reactive list for each phytochemical of the known phytochemical list. The chemicals are not particularly limited, and may be any chemical including macromolecules, heavy metals, organic compounds, elemental gasses, and other compounds. Macromolecules include lipids, proteins, and carbohydrates. The chemical database may be a public or private database, and the step may involve the sub-step of generated or collecting associations of chemicals with phytochemicals. The computerized searching required the design and execution of specific search algorithms to avoid a generation of false results. These algorithms include untrainable but not excluding the usage of trainable (natural language processing (NLP)-assisted) blocks and applied to the searches described in the below embodiments.

The method may further comprise searching a gene database comprising chemical associations with genes to generate a gene list for each chemical of each reactive chemical list. A gene is a fundamental unit of heredity in living organisms. A gene is a specific sequence of nucleotide bases in DNA (deoxyribonucleic acid) that encodes the instructions for synthesizing proteins or, in some cases, RNA molecules. Some non-limiting examples of genes include BRCA1, BRCA2, TP53, MYC, KRAS, EGFR, PTEN, ALK, BRCA1, BRCA2, TP53, MYC, KRAS, EGFR, PTEN, ALK, APP, APOE, CFTR, SODI, G6PD, HBB, HBA1, HBA2, FOXP2, MTHFR, VEGF, IL6, TNF, BCL2, P21, CDKN2A, RET, TCF7L2, DMD, FMRI, MSH2, MLHI, PMS2, XIST, OCT4, SOX2, and NANOG. The gene database may be a public or private database, and the step may involve the sub-step of generating or collecting associations of genes with chemicals.

The method may further comprise searching a pathway database comprising gene associations with physiological pathways to generate a pathway list for each gene of each gene list. A physiological pathway, also known as a biological or metabolic pathway, refers to a series of interconnected biochemical reactions that occur within a cell or organism to carry out a specific physiological function. These pathways involve enzymes, substrates, and intermediate products, and they ultimately lead to the production of important molecules or the regulation of essential processes. Some non-limiting examples of physiological pathways include glycolysis, citric acid cycle (Krebs Cycle), oxidative phosphorylation, fatty acid synthesis, beta-oxidation, pentosc phosphate pathway, urea cycle, glycogenesis, glycogenolysis, protein synthesis, nitrogen metabolism, MAPK/ERK pathway, PI3K/Akt pathway, JAK/STAT Pathway, wnt signaling pathway, notch signaling pathway, hippo pathway, TGF-beta signaling pathway, apoptosis pathway, calcium signaling pathway, immune response pathway, DNA repair pathway, and circadian rhythm pathway. The pathway database may be a public or private database, and the step may involve the sub-step of generating or collecting associations of physiological pathways with genes.

The method may further comprise searching a health effect database comprising physiological pathway associations with organism health effects to generate a health effect list for each physiological pathway of each pathway list. A health effect refers to any change in health status or condition resulting from exposure to a substance, environmental factor, lifestyle choice, or biological agent. These effects can be positive or negative and can influence physical, mental, or emotional well-being of the organism. The organism is not particularly limited and can be any living entity that exhibits the characteristics of life, including growth, reproduction, metabolism, response to stimuli, and adaptation to the environment. Most typically, the organism is a human. The health effect database may be a public or private database, and the step may involve the sub-step of generating or collecting associations of physiological pathways with organism health effects. In these embodiments, the method effectively links each phytochemical of the known phytochemical list to all of its associated chemicals, genes, physiological pathways, and health effects.

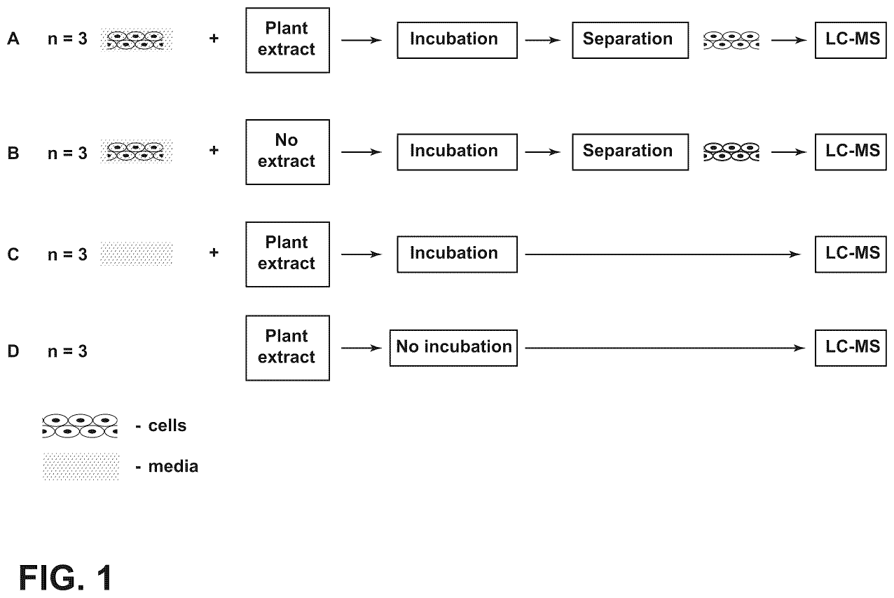

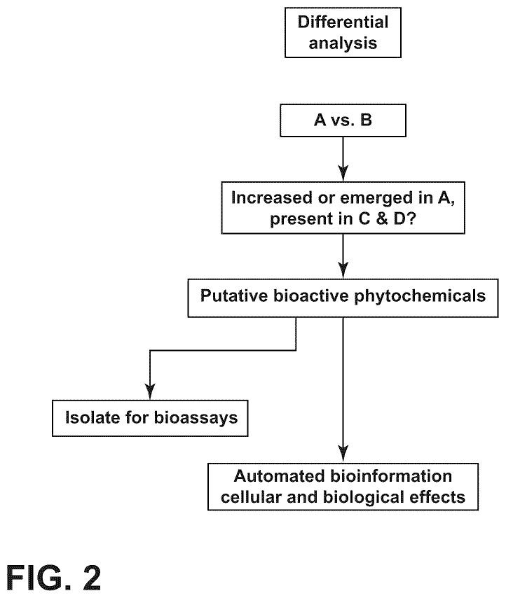

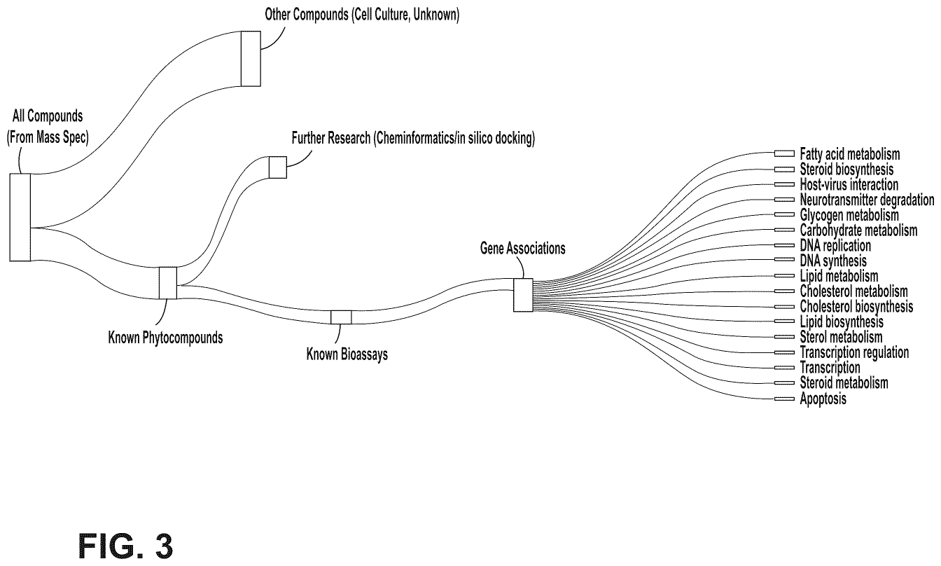

As depicted schematically in FIG. 1, the exposed cell group, the control cell group, the cell-free control, and the media-free control are measured using LC-MS. Once the relevant processing and measuring steps are conducted, the data is subjected to a differential analysis to determine the presence of phytochemicals in the exposed cell group as shown in FIG. 2. FIG. 3 schematically depicts the analysis of various compounds from loquat, including the various bioassays, gene associations, and physiological pathway associations for the loquat.

In general, assignment of a potential bioactivity to a particular compound is based on the observation of an appearance or increased concentrations of chemicals from the mixture in cellular or other biological material after coincubation of the former with cells or tissues. Therefore, the method of this disclosure is generally based on the phenomenon of binding between interacting molecules, which is an important prerequisite for biochemical and biological activities. Therefore, the method does not use assumptions driven by the result of bioassay tests and physical and chemical similarities to already known bioactive compounds. This allows a direct and confident detection of putative biologically or chemically active compounds.

The confirmation of bioactivity can be validated via isolation of a compound and its exposure to a particular bioassay. However, the method of this disclosure provides the opportunity of a high-throughput identification of putative multiple compounds using (but not limited to) mass spectrometry directly in the very first, quantitative analysis. This generally results in the detection of dozens of putative bioactive compounds. Then, it becomes possible to apply an automated (or near automated), bioinformatic meta-analysis for all these compounds and assign biological activities to them. These greatly reduces the involvement of human labor and makes the method high throughput.

In general, the detection of candidate bioactive compounds is not limited to particular bioassay(s) and already existing (i.e., published) data. Numerous compounds routinely detected using the method of this disclosure in a single experiment ensures a high-throughput discovery of unknown (i.e., new) phytochemicals.

In various embodiments, the experimental execution is based on mixing of particular cells with a complex mixture of chemicals at non-toxic concentrations and incubation of these mixes at desired conditions. Two other mixes, cells without chemicals, and chemicals without cells, are incubated at the conditions which are identical to the first mix to serve as controls in subsequent analysis. After incubation, the cells are separated from surrounding media, and both are preserved for further analysis. In certain embodiments, the analysis is executed by liquid chromatograph coupled to mass spectrometer (LC-MS). Other analysis methodologies and/or systems may also be utilized.

For LC-MS, MS is used to measure the strength of signals produced by chemicals and to identify chemicals via fragmentation analysis. The statistical analysis is applied to determine those chemicals which signals are significantly increased >1.5-fold in a pellet of cells incubated with chemicals compared to cells incubated without chemicals. Fragmentation MS2 experiments executed (but not limited) via collision induced (CID), high energy (HCD) or ultra-violet (laser) photo-dissociation (UVPD) provided an initial identification together with accurate mass and chromatography retention time. Then, chemicals are isolated from complex mixture by chromatography fractionation and subjected to additional identification procedures (e.g., nuclear magnetic resonance (NMR)) and/or used for the assessment of their biological activities in variable biological and genomic assays. The chemicals which identity as undefined are stored for further identification and assessments. The chemicals which are fully identified by their match to spectral libraries, NMR and authentic standards are submitted into a semi-automated annotation tool, which assigns to them interacting protein(s), gene(s), and partners together with cellular and biological effects.

Therefore, this disclosure provides various benefits. For example, the method provides direct detection of potentially bioactive molecules in chemical mixtures after incubation with subject biomaterial. The method provides semi-automated computerized assessment of biological activity to execute unattended assignment of biological function and roles to identified chemicals. In addition, the method can screen, assess, and identify chemicals (and their in-plant chemical modifications) which exhibit particular bioeffects but cannot be identified due to the absence of identification knowledge such as fragmentation spectrum, two- or three-dimensional molecular architecture (i.e., structure), atomic composition, etc. Further, the method can screen, assess, and identify compounds, which were known previously, but now provide newly discovered biological activity. Moreover, the method can be used to detect compounds in a chemical mix (e.g., in plant extracts) for the first time.

As noted above, various aspects of the method and corresponding system can be varied. Some of these possible variations are outlined below. One of skill will understand other variations may be possible, and such can be determined via routine knowledge or experimentation.

Variations in analytical methods of detection include UV/Vis, PDA, electrochemical or potentiometric, molecular interactions (e.g., affinity or immunoaffinity), separation (e.g., electrophoresis, chromatography and ion mobility separations), fractionation (e.g., chromatography or by other devices or methods), mass spectrometry and other methods for signal detection (e.g., ESI, APCI, CI, MALDI, or photo-ionization), formula calculations, fragmentation (e.g., CID, HCD, UVPD, or EAD execution), and identification (e.g., interpretation of MS fragmentation of any sort, identification by authentic or labelled standards, by NMR or X-Ray,) or other molecular identification techniques. Assignment of cellular and/or biological functions, including bioassays (e.g., in-vitro cellular tests, binding assays, flow cytometry, GNS, or RT-PCR).

Referring now to the Figures, FIGS. 1 and 2 are flow charts of an embodiment of HTS analysis according to this disclosure. Cells in a suitable growth media is co-incubated with a plant extract or a mix of chemicals disregarding their complexity and origin. Extraction of plant material is understood in the art, e.g., as described in U.S. Pat. No. 8,202,556 to Rana et al. While triplicate testing (i.e., n=3) is shown, it is to be appreciated that single tests or a plurality of tests can be performed (including just 2 or 4 or more). Incubation techniques are also understood in the art. In general, bioactive chemicals should adhere or penetrate cells and increase in concentration in cell pellet of treated (A) vs. untreated (B) ones. Such chemicals, if they are detected in plant extract without incubation (D) or emerge during an incubation with media (C) as chemical modification of plant phytochemicals are designated as putative bioactive phytochemical. These ones are the subject of further identification, validation, isolation, and bio- or genetic assays.

FIG. 3 depicts an automated bioinformatic data analysis, which annotates compounds detected in mass spectrometry (MS) analysis. Twenty-seven compounds which signal increased at least 2-fold (p<0.05) in fibroblasts exposed to the extract of loquat leaves compared to non-exposed cells (dark blue vertical bar on the left). The putative bioactive compounds can be separated into three categories: a) Unknown-Unknown (light blue, on the top), which identity is not known; b) Known-unknown, which identity is known, but biological or chemical activity is not (violet bar, in the middle); and c) known-known, i.e., compounds identified and reported as bioactive in repositories of bioassays such as (but not limited to) PubChem. Gene targets were retrieved from the list of positive assays and submitted into the David database for functional annotation and meta-analysis of health benefits.

### INDUSTRIAL APPLICABILITY

This disclosure provides new and useful methods for screening compounds, and more specifically, to high throughput screening (HTS) methods for the detection of bioactive compounds (or molecules) in complex chemical mixtures, such as in plants, in plant extracts, etc. Thus, this disclosure also provides bioactive compounds, molecules, or phytochemicals screened via the methods of this disclosure. In addition, this disclosure provides compositions and uses of such bioactive compounds, molecules, or phytochemicals.

Overall, the methods herein provide for high-throughput, confident discovery of multiple chemical entities which are capable to exert biological activity in complex mixtures. Identification and automated annotation of their biological effects in animals and humans is also possible.

The following examples, illustrating the methods and systems of this disclosure, are intended to illustrate and not to limit the disclosure.

### EXAMPLES

Utilizing the methods of this disclosure, such as that illustrated in FIG. 1, rosemary (Rosemarinus officinalis) is analyzed. Tables 1 and 2 below shows potentially bioactive compounds detected by HTS of extracts of rosemary, e.g., from leaves and/or aerial parts of the plant. Table 2 shows potentially bioactive compounds detected by HTS in extracts of Rosemary officinalis. The list consists of validated (against standards via fragmentation analysis) and not validated (“Identified by HRMS only”) phytochemicals.

TABLE 1

| Name | PBA001 | PBA002 |
|---|---|---|
| Monoisotopic Mass | 302.1883 | 300.2088 |
| RT | 26.6 | 38.12 |
| Formula | C₁₉H₂₆O₃ | C₂₀H₂₈O₂ |
| Precursor | 303.1957 | 301.216 |
| Fragment 1 | 191.1066 | 205.1222 |
| Fragment 2 | 215.1066 | 231.138 |
| Fragment 3 | 149.0597 | 219.138 |
| Fragment 4 | — | 148.0518 |

TABLE 2

| | | | | | ID'ed | ID'ed |
|---|---|---|---|---|---|---|
| | | | | RT | by LC- | by |
| Name | [M + H]⁺¹ | [M + H]⁻¹ | Area | (min) | MSMS | HRMS |
| Pectolinarigenin? | 315.0860 | — | 7.0 × 10⁹ | 10.29 | No | Yes |
| Formestane? | 303.1955 | — | 1.0 × 10⁸ | 16.20 | No | Yes |
| Mosloflavone? | 299.0914 | — | 5.0 × 10⁹ | 14.72 | No | Yes |
| 18-β-glycyrrhetinic | 471.3469 | — | 1.5 × 10⁹ | 18.43 | No | Yes |
| Acid? | | | | | | |
| Retinoic acid? | 301.2160 | — | 8.0 × 10⁹ | 20.45 | No | Yes |
| Scrophulein | 315.0863 | — | 7.0 × 10⁹ | 26.83 | No | Yes |
| (cirsimaritin)? | | | | | | |
| Rethymicin? | 329.1019 | — | 2.0 × 10⁹ | 34.42 | No | Yes |
| Rosmarinic Acid | — | 359.0768 | 7.0 × 10⁹ | 6.56 | Yes | No |
| Genkwanin | 285.0755 | — | 4.0 × 10⁹ | 12.09 | Yes | No |
| Carnosol | 331.1901 | — | 4.5 × 10⁹ | 15.86 | Yes | No |
| Carnosic Acid | — | 331.1915 | 1.4 × 10⁹ | 19.72 | Yes | No |
| 12-O-methylcarnosic | — | 345.2067 | 1.0 × 10⁹ | 20.44 | Yes | No |
| Acid | | | | | | |
| Ursolic Acid | — | 455.3531 | 4.0 × 10⁸ | 22.26 | Yes | No |
| Oleanolic Acid | — | 455.3531 | 1.0 × 10⁸ | 22.29 | Yes | No |
| Betulinic Acid | — | 455.3531 | 3.0 × 10⁹ | 21.66 | Yes | No |

The terms “comprising” or “comprise” are used herein in their broadest sense to mean and encompass the notions of “including”, “include”, “consist(ing) essentially of”, and “consist(ing) of”. The use of “for example”, “e.g.”, “such as”, and “including” to list illustrative examples does not limit to only the listed examples. Thus, “for example” or “such as” means “for example, but not limited to” or “such as, but not limited to” and encompasses other similar or equivalent examples. The term “about” as used herein serves to reasonably encompass or describe minor variations in numerical values measured by instrumental analysis or as a result of sample handling. Such minor variations may be in the order of ±0-10, ±0-5, or ±0-2.5, % of the numerical values. Further, the term “about” applies to both numerical values when associated with a range of values. Moreover, the term “about” may apply to numerical values even when not explicitly stated.

Generally, as used herein a hyphen “-” or dash “-” in a range of values is “to” or “through”; a “>” is “above” or “greater-than”; a “≥” is “at least” or “greater-than or equal to”; a “<” is “below” or “less-than”; and a “≤” is “at most” or “less-than or equal to”. On an individual basis, each of the aforementioned applications for patent, patents, and/or patent application publications, is expressly incorporated herein by reference in its entirety in one or more non-limiting embodiments.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is also to be understood that any ranges and subranges relied upon in describing various embodiments of the present disclosure independently and collectively fall within the scope of the appended claims and are understood to describe and contemplate all ranges including whole and/or fractional values therein, even if such values are not expressly written herein. One of skill in the art readily recognizes that the enumerated ranges and subranges sufficiently describe and enable various embodiments of the present disclosure, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present disclosure has been described herein in an illustrative manner, and it is to be understood that the terminology which has been used is intended to be in the nature of words of description rather than of limitation. Many modifications and variations of the present disclosure are possible considering the above teachings. The present disclosure may be practiced otherwise than as specifically described within the scope of the appended claims. The subject matter of all combinations of independent and dependent claims, both single and multiple dependent, is herein expressly contemplated.

1. A screening method for the high-throughput detection of phytochemicals, the method comprising:
exposing an extract cell group to a plant extract comprising one or more phytochemicals to form an exposed cell group;
measuring and comparing exposed signals associated with the one or more phytochemicals and generated by the exposed cell group to control signals generated by a control cell group to generate a signal list consisting of exposed signals that satisfy an inclusion condition;
comparing the exposed signals of the signal list to a list of pre-determined phytochemical signatures, each phytochemical signature associated with at least one individual phytochemical, to generate a known phytochemical list and/or an unknown phytochemical list, wherein the known phytochemical list consists of phytochemicals having phytochemical signatures substantially matching one of the exposed signals of the signal list, and wherein the unknown phytochemical list consists of any of the one or more phytochemicals that correspond with the exposed signals that do not substantially match one of the exposed signals of the signal list; and
searching a chemical database comprising phytochemical associations with chemicals to generate a reactive chemical list for each phytochemical of the known phytochemical list.

2. The method of claim 1, further comprising searching a gene database comprising chemical associations with genes to generate a gene list for each chemical of each reactive chemical list.

3. The method of claim 2, further comprising searching a pathway database comprising gene associations with physiological pathways to generate a pathway list for each gene of each gene list.

4. The method of claim 3, further comprising searching a health effect database comprising physiological pathway associations with organism health effects to generate a health effect list for each physiological pathway of each pathway list.

5. The method of claim 1, further comprising culturing a plurality of cells and subdividing the plurality of cells into the extract cell group and the control cell group.

6. The method of claim 1, wherein the extract cell group and the control cell group are each disposed within a cell media.

7. The method of claim 6, further comprising preparing a cell-free control comprising the plant extract and the cell media and a media-free control comprising the plant extract, wherein the cell-free control and media-fee control are substantially free of the plurality of cells, and wherein the media-free control is substantially free of the cell media.

8. The method of claim 7, further comprising comparing the exposed signals to extract signals of the media-free control to associate the exposed signals with the one or more phytochemicals.

9. The method of claim 7, further comprising incubating the extract cell group, the control cell group, and the cell-free control.

10. The method of claim 6, further comprising separating the extract cell group and the control cell group from the respective cell media.

11. The method of claim 1, wherein the inclusion condition is satisfied when an individual exposed signal is greater than a corresponding individual control signal.

12. The method of claim 1, wherein the exposed signals and the control signals are mass spectroscopy and/or nuclear magnetic resonance signals.

13. The method of claim 1, wherein comparing the exposed signals of the signal list to a list of pre-determined phytochemical signatures comprises generating the unknown phytochemical list, and wherein the method further comprises isolating any of the one or more phytochemicals of the unknown phytochemical list from the exposed cell group and/or plant extract.

14. The method of claim 13, wherein isolating any of the one or more phytochemicals of the unknown phytochemical list comprises using chromatography to separate any of the one or more phytochemicals of the unknown phytochemical list from the exposed cell group and/or plant extract.

15. The method of claim 13, further comprising characterizing any of the one or more phytochemicals of the unknown phytochemical list.

16. The method of claim 1, wherein comparing the exposed signals of the signal list to a list of pre-determined phytochemical signatures comprises generating the known phytochemical list, and wherein the method further comprises isolating any of the one or more phytochemicals of the known phytochemical list from the exposed cell group and/or plant extract.

17. The method of claim 16, wherein isolating any of the one or more phytochemicals of the known phytochemical list comprises using chromatography to separate any of the one or more phytochemicals of the known phytochemical list from the exposed cell group and/or plant extract.
